(19) United States
(12) Patent Application Publication
(10) Pub. No.: US 2025/0069732 A1
(43) Pub. Date: Feb. 27, 2025

(54) MULTI-SIDED MATCH MAKING PLATFORMS

(71) Applicant: State Farm Mutual Automobile Insurance Company, Bloomington, IL (US)

(72) Inventors: Tim G. Sanidas, Bloomington, IL (US); Edward P. Matesevac, Normal, IL (US); Anthony Nathan Noel, Bloomington, IL (US); Chris Kawakita, Normal, IL (US); Garrick Douglas Gabbrants, Bloomington, IL (US)

(21) Appl. No.: 18/946,048

(22) Filed: Nov. 13, 2024

Related U.S. Application Data
(63) Continuation of application No. 16/918,876, filed on Jul. 1, 2020, now Pat. No. 12,170,143.
(60) Provisional application No. 62/870,528, filed on Jul. 3, 2019.

(51) Int. Cl. G16H 40/20; G06N 20/00; G16H 40/67

(52) U.S. Cl. CPC G16H 40/20; G06N 20/00; G16H 40/67

(57) ABSTRACT

A multi-sided match making (“MMM”) computer system including at least one processor in communication with at least one memory device for matching consumers to providers is provided. The at least one processor is configured to: (i) receive registration data from a user, (ii) receive user data from at least one of a sensor and a mobile device associated with the user, (iii) analyze the registration data and the user data, (iv) determine a need based upon the analyzed registration and user data, (v) transmit the determined need to at least one caregiver associated with the user, and (vi) match the user to at least one provider based upon the determined need, wherein the provider is at least one of the caregiver, another caregiver, and a service, and wherein the provider is able to meet the determined need for the user.

### CROSS-REFERENCE TO RELATED APPLICATIONS

This application is a continuation of, and claims the benefit of priority to, U.S. patent application Ser. No. 16/918,876, filed Jul. 1, 2020, entitled “MULTI-SIDED MATCH MAKING PLATFORMS,” which claims the benefit of priority to U.S. Provisional Patent Application Ser. No. 62/870,528, filed Jul. 3, 2019, entitled “MULTI-SIDED MATCH MAKING PLATFORMS,” the entire contents and disclosures of which are hereby incorporated herein by reference in their entirety.

### FIELD OF THE INVENTION

The present disclosure relates to multi-sided platforms and, more particularly, to systems and methods for using a multi-sided platform to make matches based upon a person's determined needs.

### BACKGROUND

At least some conventional computer networks (e.g., matching platforms) have enabled consumers to be matched with providers. However, conventional systems usually merely match consumers with providers (e.g., one-sided platforms or two-sided platforms), and may not provide additional functionality. Further, in the known systems, consumers may have to determine a specific need before being matched with a provider. Known systems may have other drawbacks as well.

### BRIEF SUMMARY

The present embodiments may relate to systems and methods for electronically determining one or more needs of a user and matching the user to providers based upon the determined needs. The system may include a multi-sided match making computing device, one or more third party servers, one or more client devices, one or more sensor servers, and/or one or more databases.

In one aspect, a multi-sided match making (“MMM”) computer system for matching consumers to providers is provided, and the MMM computer system includes at least one processor in communication with at least one memory device. The at least one processor may be configured to: (i) receive registration data from a user, (ii) receive user data from at least one of a sensor and a mobile device associated with the user, wherein the user data is generated by the at least one of the sensor and the mobile device in response to an action carried out by the user, (iii) analyze the registration data and the user data, (iv) determine a need based upon the analyzed registration and user data, (v) transmit the determined need to at least one caregiver associated with the user, and/or (vi) match the user to at least one provider based upon the determined need, wherein the provider is at least one of the caregiver, another caregiver, and a service, and wherein the provider is able to meet the determined need for the user. The MMM computer system may include additional, less, or alternate functionality, including that discussed elsewhere herein.

In another aspect, a computer-implemented method for matching consumers to providers using a multi-sided match making (“MMM”) computer system including at least one processor in communication with at least one memory device is provided. The method may include: (i) receiving registration data from a user, (ii) receiving user data from at least one of a sensor and a mobile device associated with the user, wherein the user data is generated by the at least one of the sensor and the mobile device in response to an action carried out by the user, (iii) analyzing the registration data and the user data, (iv) determining a need based upon the analyzed registration and user data, (v) transmitting the determined need to at least one caregiver associated with the user, and/or (vi) matching the user to at least one provider based upon the determined need, wherein the provider is at least one of the caregiver, another caregiver, and a service, and wherein the provider is able to meet the determined need for the user. The method may include additional, less, or alternate actions, including those discussed elsewhere herein.

In yet another aspect, at least one non-transitory computer-readable media having computer-executable instructions thereon, wherein when executed by at least one processor of a multi-sided match making (“MMM”) computer system causes the at least one processor of the MMM computer system to: (i) receive registration data from a user, (ii) receive user data from at least one of a sensor and a mobile device associated with the user, wherein the user data is generated by the at least one of the sensor and the mobile device in response to an action carried out by the user, (iii) analyze the registration data and the user data, (iv) determine a need based upon the analyzed registration and user data, (v) transmit the determined need to at least one caregiver associated with the user, and/or (vi) match the user to at least one provider based upon the determined need, wherein the provider is at least one of the caregiver, another caregiver, and a service, and wherein the provider is able to meet the determined need for the user. The instructions may direct additional, less, or alternate functionality, including that discussed elsewhere herein.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and are instrumentalities shown, wherein:

FIG. 1 illustrates a flow chart of an exemplary process of matching users to providers for a multi-sided match making system;

FIG. 2 illustrates an exemplary multi-sided match making computer system for electronically matching users to providers in accordance with the present disclosure;

FIG. 3 illustrates an exemplary configuration of an exemplary user computing device that may be used in the multi-sided match making computer system illustrated in FIG. 2;

FIG. 4 illustrates an exemplary configuration of an exemplary server computing device that may be used in the multi-sided match making computer system illustrated in FIG. 2;

FIG. 5 illustrates a flow chart of an exemplary computer-implemented method implemented by the exemplary multi-sided match making computer system shown in FIG. 2;

FIG. 6 illustrates a diagram of components of one or more exemplary computing devices that may be used in the multi-sided match making computer system shown in FIG. 2; and

FIG. 7 illustrates an exemplary computer-implemented method associated with a multi-sided caregiver platform (MSCP) computer system as described herein.

The Figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION

The present embodiments may relate to, inter alia, systems and methods for electronically determining one or more needs of a user and matching the user to one or more providers based upon the determined needs of the user. In one exemplary embodiment, the process may be performed by a multi-sided match making (“MMM”) computer system (also referred to herein as a “NMM platform” and a “MMM server”). In another embodiment, the process may be performed by a multi-sided caregiver platform (“MSCP”) computer system, which may be configured to perform steps that are substantially similar to those described herein for the MMM computer system (e.g., the MMM computer system may include functionality substantially similar to the functionality of the MSCP computer system).

As described below, the systems and methods described herein may leverage sensor and mobile device data to determine a need of a user and match the user to one or more providers (e.g., caregivers and/or services) able to take care of the determined need for the user. The systems and methods described herein may learn the needs of the user such that the systems and methods described herein may automatically make arrangements with an appropriate one of the providers on behalf of the user without the user specifically initiating the process of having a need met. In some embodiments, the user may be a senior user who needs extra help going about his or her daily routine (e.g., rides to appointments, refills on their prescriptions, monitoring of activity, and completion of tasks, like taking their medicine). Caregivers (e.g., family members, friends, healthcare professionals, etc.) of the user may not be able to help the user every day, and friction between different caregivers may result if one or more caregivers help the user more often than other caregivers. Accordingly, the systems and methods described herein ensure that the needs of the users are met with little input from the user, and may reduce friction between different caregivers by effectively matching the user with the provider who is best able to take care of the user's determined need.

### Exemplary User Data Collection

In the exemplary embodiment, a multi-sided match making (“MMM”) service (e.g., provided by a multi-sided match making server) may leverage user data (e.g., sensor data, mobile device data, and registration data) to determine the needs of a user, match the user to one or more providers, and/or automatically make arrangements with the providers for the user. In the exemplary embodiment, the user may register for the multi-sided match making service provided by a multi-sided match making (“MMM”) server through a mobile device associated with the user (e.g., through a multi-sided match making application on the mobile device) or through any other suitable device. As the user registers for the MMM service, the user may provide registration data to the MMM server for the MMM server to store in a memory device associated with the MMM server. Registration data may include, for example, age, birthdate, height, weight, medical history, preferred providers, and any other data associated with the user.

Further, in the exemplary embodiment, the user may enable the MMM server to receive data from servers associated with sensors that are associated with the user. For example, sensors may include mobile device sensors (e.g., GPS, speakers, and microphone), smart home device sensors (e.g., AMAZON ALEXA, GOOGLE HOME, NEST devices, and RING devices), and wearable device sensors (e.g., FITBIT and APPLE WATCH). Sensors may also include autonomous vehicle sensors, smart vehicle sensors, and/or other vehicle sensors relating to telematics (e.g., cornering, braking, acceleration, etc.) of a vehicle, and the sensors may be directly coupled to the vehicle (e.g., back-up cameras, proximity sensors, etc.), and/or associated with the mobile device (e.g., sensors, including GPS, accelerometers, gyroscopes, etc., associated with a mobile telematics application) when the vehicle is being driven. Sensors may also be smart infrastructure sensors (e.g., streetlight, street, and traffic sensors), and/or smart building sensors (e.g., building cameras, building entrance/exit sensors, and utility sensors). The user may also enable the MMM server to receive data (e.g., notifications, reminders, and calendar data) from the mobile device of the user.

The data received by the MMM server from the sensors and/or the mobile device associated with the user may be considered user data, and the user data may be generated by the sensors and/or the mobile device in response to an action taken by the user when the user interacts with the sensor and/or the mobile device. Data from the sensors may include, for example, a GPS location of the user, conversations the user has over the mobile device or evidence of patterns of conversations without requiring verbatim collection of conversation content, questions the user asks the smart home device, driving scores and/or travel data from the vehicle and/or the mobile device, daily activity data, sleeping pattern data, movement data, physical condition data, and/or vital data from the wearable device, home utility/telematics data (e.g., gas, water, and electric usage and occupancy data) from the building and/or smart home device sensors, and any other data from sensors that may be useful to the MMM server in determining a need of the user. Data from the mobile device may include, for example, notifications sent to the user, third party application data (e.g., doctor and pharmacy applications relating to, for example, appointment scheduling and prescription refill scheduling), a calendar of the user, phone usage data (e.g., how often the phone is used and/or how many phone calls/text messages are sent out in a day), social media data (e.g., social interaction data and response times to messages/comments), and any other data from the mobile device that may be useful to the MMM server in determining a need of the user.

Once the user is registered and the MMM server is able to receive data from the sensors and the mobile device associated with the user, the MMM server may begin collecting and storing user data from the sensors and/or from the mobile device.

### Exemplary Need and Abnormal Condition Determination

In the exemplary embodiment, the MMM server may analyze the received user and registration data to determine a need and/or an abnormal condition of the user. For example, if the user schedules a doctor's appointment on the mobile device associated with the user and the MMM server receives this scheduled appointment as user data from the mobile device, the MMM server may determine that the need for the user is that the user needs to get to the user's scheduled doctor's appointment. Further, the MMM server may analyze the registration data from the user and determine that the user is unable to drive. Thus, the MMM server may determine that the need for the user is a ride to their scheduled doctor's appointment.

To analyze the data, the MMM server may input the received user and registration data into a machine learning algorithm and/or a machine learning program, as described in further detail below. In the exemplary embodiment, the machine learning algorithm and/or program may be trained to identify needs and/or abnormal conditions of the user. The machine learning algorithm and/or program may further “learn” from the user data and predict needs and/or abnormal conditions associated with the user before the need and/or the abnormal condition arise.

In analyzing the received user and registration data, the MMM server may discover patterns in the user's routine. The MMM server may determine that the user picks up a maintenance medication around a certain day of every month, and the MMM server may determine this from notifications (e.g., a text message) from the pharmacy on the certain day and/or a GPS location of the user on the certain day. Accordingly, the MMM server may determine that picking up the medication from the pharmacy around the certain day every month is a pattern in the routine of the user, and the MMM server may store this discovered pattern in memory associated with the MMM server.

In analyzing the received user and registration data, the MMM server may further discover pattern disruptions in the user's routine. For example, in reference to the above, the MMM server may receive user data including multiple notifications from the pharmacy that the user needs to pick up their medication, and the MMM server may determine that the user forgot to pick up their medication around the certain day this month. Accordingly, the MMM server may determine that the user forgetting to pick up their medication was a pattern disruption. If the MMM server recognizes multiple pattern disruptions in the user's routine, the MMM server may determine, for example, that a need of the user, in addition to their medicine, is to see a doctor to have the memory of the user evaluated.

Further, for example, the MMM server may receive and store vehicle telematics data associated with the user as user data. The MMM server may determine that the user has a pattern of being a good driver because of their generally slow acceleration and braking speeds. If the MMM server receives vehicle telematics data that the braking speeds of the driver are becoming faster and faster, the MMM server may determine that there is a pattern disruption and/or abnormal condition in the driving behavior of the user. From the determined pattern disruption in the user's driving patterns, the NM server may determine a need of the user (e.g., that the user needs to get their eyesight checked and/or that the user needs to have another driving test).

### Exemplary Match Making and Provider Arrangements

In the exemplary embodiment, the MMM server may match the user to a provider based upon the determined need of the user. Providers may be, for example, caregivers (e.g., family members, friends, healthcare professionals) and third parties that provide a service (e.g., ride-sharing services, pharmacy delivery services, medical services, etc.). In the exemplary embodiment, one or more caregivers may be associated with the user.

The caregivers may be able to access the MMM application (e.g., on a mobile device associated with the caregivers), and the caregivers may be able to access the determined needs and/or abnormal conditions of the user through the MMM application and information related to the user from the MMM server. Further, the caregivers may be able to provide the MMM server with the schedule of the caregivers (e.g., work and activity schedules) and tasks that the caregivers carry out for the user (e.g., one caregiver typically picks up the user to go to an aerobics class on Monday evenings, a second caregiver typically picks up a prescription for the user in the first week of each month, and/or a third caregiver typically takes the user to their doctor's appointments when the third caregiver is not working). Further, the caregiver may allow the MMM server to access their location data (e.g., through a GPS sensor of a mobile device associated with the caregiver).

The information provided by the caregivers may allow the MMM server to determine which caregiver, if any, is available to take care of a need of the user and/or remedy an abnormal condition of the user when the MMM server determines the need and/or abnormal condition. Accordingly, the MMM server may match the user with the caregiver that satisfies scheduling criteria input by the caregivers when a need and/or abnormal condition is determined by the MMM server. Further, the MMM server may match the user with the caregiver who is in closest proximity to the location of the user, for example, if the MMM server determines a more urgent need and/or abnormal condition of the user.

Further, in the exemplary embodiment, the MMM server may match the user with a third party that provides a service based upon the determined need and/or abnormal condition of the user. For example, the third party services may be used if the MMM server determines that no caregivers are available to take care of the need and/or abnormal condition of the user. Further, for example, the caregivers may input (e.g., into the MMM application) that the caregivers prefer that the MMM server matches the user with third party services to take care of the need and/or abnormal condition of the user before the MMM server matches the user with the caregiver to take care of the need and/or abnormal condition of the user. Accordingly, the MMM server efficiently matches the user to the provider that satisfies user- and/or caregiver-specified criteria in order of preference of the user and/or caregiver to take care of the need and/or abnormal condition of the user.

After the MMM server matches the user with the provider to take care of the need and/or abnormal condition of the user, the MMM server may further automatically make arrangements with the provider on behalf of the user. For example, if the user needs to pick up a prescription at a certain time on a certain date, the MMM server may determine that none of the caregivers are available at the certain time and date, and the MMM server may then schedule a ride (e.g., through UBER or LYFT ride sharing services) for the user to the pharmacy at the certain time and date. Once the third party receives the arrangement, the third party may send a confirmation (e.g., through an application associated with the third party and/or through the MMM application) to the MMM server that the need of the user will be taken care of by the third party. Further, if the MMM server makes an arrangement with a caregiver, the caregiver may also send a confirmation (e.g., through MMM application) to the MMM server that the caregiver will take care of the determined need of the user.

Once the MMM server receives the confirmation of the generated arrangements from the providers, the MMM server may send a notification to the user notifying the user of the confirmed arrangement. The notification to the user may include, for example, a text message, a push notification, an email, a phone call, and/or any other suitable notification method.

In the exemplary embodiment, the MMM server may be configured to receive feedback from the user concerning the generated arrangements. The user may, through the MMM application, rate the arrangements and/or the provider and provide comments or feedback about the arrangements and/or the provider. Further, the MMM server may use the user feedback in making further arrangements with third parties for the user in the future. For example, if the arranged third party service was on time and friendly, the user may provide that feedback to the MMM server and rate the third party service with five stars. Further, if the MMM server determines that the user needs a third party provider again, the MMM server may request the same third party for the determined need based upon the good user feedback.

### Exemplary Multi-Sided Caregiver Platform Functionality

In one aspect, the present embodiments enable senior patients, family, and professional caregivers to coordinate interaction and care, keep track of health progress and health activity, store health information and other important documents, access educational information, manage transportation, and navigate various community resources. A multi-sided caregiver platform (“MSCP”) for seniors may include and/or utilize a portal and mobile application may facilitate multiple caregivers to engage third parties, such as plumbers, repairmen, doctors, or other service providers. In some embodiments, the MSCP is substantially similar to, and works in substantially the same way as MMM server, described above. In one embodiment, multiple seniors or other consumers may engage multiple service providers for services and/or products. In other embodiments, several seniors or customers may engage a single provider, or vice-versa. In other words, more than a one-to-one customer/service provider interaction(s) may be facilitated by the MSCP.

The present embodiments may use artificial intelligence and/or machine learning techniques on data, such as the data and types of data discussed elsewhere herein (such as mobile device data, mobile device sensor data, smart or interconnected home data, smart or interconnected home sensor data, smart vehicle data, smart vehicle sensor data, vehicle telematics data (such as vehicle location, speed, acceleration, cornering, deceleration, and braking data), home telematics data (such as electricity and water usage data, and occupancy and movement data), and/or other types of sensor data, including digital or other image data) to identify unmet needs of seniors or other customers, and then identify service providers to satisfy those needs. In one embodiment, the data and types of data, including sensor data, may be transformed into consumable data, or data that is otherwise digestible, for machine learning algorithms.

The present embodiments may also identify and/or schedule individual transactions and allow for electronic or other payment from a senior or other customer to a service provider. Each individual transaction may be identified and/or associated with a smart contract, and each individual transaction and/or smart contract may be placed or stored in a blockchain that may be dedicated to the customer and/or service provider, or both, as described below.

In one embodiment, whether a senior or other customer is taking prescribed medication on time and/or as prescribed may monitored. Machine learning algorithms or other artificial intelligence may identify a pattern of forgetfulness or identify the onset of forgetfulness, and if so, an electronic notification may be generated and transmitted to a mobile device of a family member or other caregiver. Machine learning may also identify when prescriptions need to be refilled, and if so, an electronic notification may be sent to a pharmacy and/or family member or other caregiver.

In some embodiments, active and/or passive monitoring of a senior may be utilized. For instance, smart home sensors and/or other sensors mounted about a home may employ active and/or passive monitoring within a home. Machine learning algorithms or other artificial intelligence may be utilized on the active and/or passive monitoring sensor data to determine irregular patterns, and if so, generate electronic notifications for family members and other caregivers.

### Exemplary Smart Contracts and Blockchain

A blockchain is a distributed database that maintains a continuously-growing list of ordered records, known as blocks. Each block may contain at least a timestamp and a link to the previous block in the chain. The link to the previous block may be a hash of the previous block. For example, in the case of a service contract, the first block may contain the initial contract between a user and a service provider. The second block may contain a modification to the contract that was requested by the service provider and approved by the user. The second block may contain a hashed copy of the first block as well. The third block may contain one or more additional terms for the service contract and a hashed copy of the second block. This continues on with each block adding on to the next while containing a hash of the previous blocks in the blockchain.

To ensure the security of the information contained in the blockchain, copies of the blockchain may be distributed across multiple computer devices, known as nodes. These nodes maintain the blockchain, update the blockchain when changes occur, and ensure the stability of the blockchain itself. In some embodiments, nodes may also be used to calculate the hash of the previous blocks. As the blockchain grows, the processing power needed to calculate the hash of the previous blocks grows as well. In these embodiments, the processing of the hash may be distributed over multiple computer devices to improve the speed of processing and/or to not overburden the hashing processor. When a node processes (hashes) a block, that node is known as a miner, where the action of validating and hashing the block is also known as mining.

Accordingly, in the exemplary embodiment, once the user and the caregiver and/or service provider are matched, the MMM server or MSCP server may generate a smart contract. The smart contract may include at least one of details of the match (also called the “transaction” herein), any restrictions on the transaction, details of any insurance policies covering the parties of the transaction, and payment, such as payment card and/or checking account information. In the exemplary embodiment, the smart contract may be stored in a blockchain ledger that is distributed among a plurality of participants (also known as nodes). The nodes may be capable of communicating with the MMM server. Each match may cause the MMM server to generate a new smart contract, and each smart contract may be stored in its own blockchain ledger. As the smart contract is modified or additional information is added to the smart contract, such as completion of the transaction, another block may be added to the blockchain of the smart contract. Further, in some embodiments, each transaction between users and providers may cause the MSCP to generate a smart contract, and each transaction and/or smart contract may be stored on the blockchain.

### Care Support Dashboard

In one aspect, the MSCP computer system and/or MMM computer system may include a care support dashboard for the senior and/or caregivers. The care support dashboard may graphically or through text represent various types of information about the senior. For instance, the dashboard may provide a graphical representation of the senior's gait over time. The senior's gait may be determined, for instance, by analysis of home sensor (image) data, wearable sensor data, and/or mobile device data.

The care support dashboard may also graphically represent sleep patterns as determined from sensor and/or image data. For instance, REM sleep, deep sleep, light sleep, and/or interrupted sleep patterns may be visually depicted on the dashboard.

The care support dashboard may also graphically depict a breakdown of the senior's activities, such as eating, bathing, exercise, movement, travel, socializing, appointments, etc. The care support dashboard may include an image of the senior and depict their physical conditions, such as heart rate. The care support dashboard may also include a display of upcoming activities or appointments. The dashboard may display other types of information, including those discussed elsewhere herein.

### Exemplary Process for Match Making

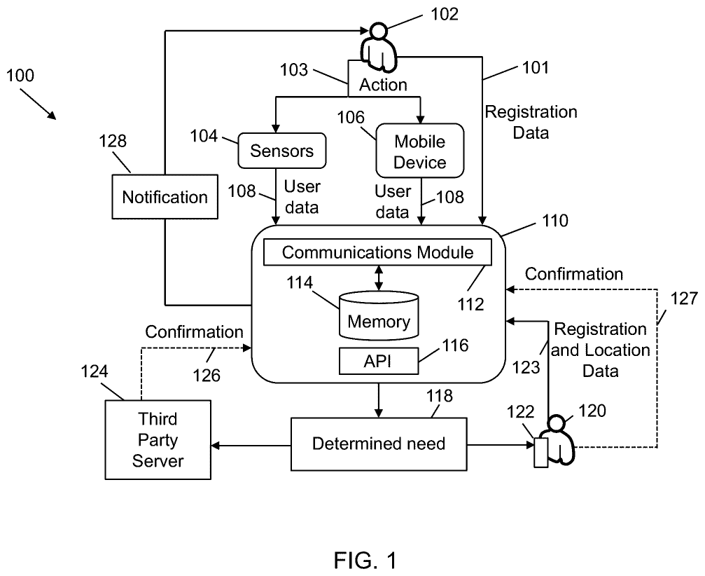

FIG. 1 illustrates a flow chart of an exemplary computer-implemented process 100 for matching a user with a provider based upon one or more determined needs and/or abnormal conditions of a user 102. In the exemplary embodiment, user 102 may register for a multi-sided match making service (e.g., provided by a multi-sided match making server 110) through a user interface (not specifically shown) of a mobile device 106 associated with user 102, which may enable communication with a communications module 112 of multi-sided match making (“MMM”) server 110. Registration data 101 for user 102 may be transmitted to MMM server 110 and may include personal information (e.g., birthday, age, height, etc.), health information (e.g., health history, current health status, etc.), and other information relating to user 102 (e.g., user's primary care doctor, ability to drive, preferred pharmacy, etc.). Registration data 101 may be stored in memory 114 of MMM server 110. In the exemplary embodiment, process 100 may be associated with matching elderly (also called “senior” herein) and/or dependent users 102 with one or more providers.

In the exemplary embodiment, once user 102 is registered for the multi-sided match making service, user 102 may be actively or passively monitored. User 102 may be monitored by sensors 104 and/or a mobile device 106 associated with user 102. Sensors 104 may include smart home devices and wearable devices. In other embodiments, sensors 104 may also include smart building sensors, aerial device sensors, smart vehicle sensors, autonomous or semi-autonomous vehicle sensors, and/or other sensors. When user 102 carries out an action 103, sensors 104 and/or mobile device 106 may detect action 103. For example, if user 102 asks their smart home device a question, sensor 104 detects this action 103 (e.g., the asking of the question). Further, for example, if user 102 makes an appointment on mobile device 106 through an application, mobile device 106 detects this action 103 (e.g., the making of the appointment).

In response to detecting action 103, sensors 104 and/or mobile device 106 may generate user data 108 related to action 103 and may transmit generated user data 108 to MMM server 110. In the exemplary embodiment, user data 108 may be transmitted to MMM server 110 from sensors 104 and/or mobile device 106. In some embodiments, MMM server 110 may be associated with sensors 104 and/or mobile device 106 and may actively retrieve user data 108 from memory 114 of MMM server 110.

In the exemplary embodiment, user data 108 may be retrieved and stored in memory 114 of MMM server 110. MMM server 110 may analyze user data 108 and generate a determined need 118 based upon analyzed user data 108. In some embodiments, determined need 118 also includes a determined abnormal condition of user 102. For example, if user data 108 is related to user 102 making a doctor's appointment, and MMM server 110 retrieves (e.g., from registration data 101 in memory 114) that user 102 cannot drive, MMM server may determine that user 102 needs a ride to the scheduled doctor's appointment. NMM server 110 may transmit determined need 118 to a mobile device 122 (e.g., through a notification to mobile device 122 and/or through a multi-sided match making application on mobile device 122) associated with a caregiver 120 of user 102. Further, MMM server 110 may transmit determined need 118 to a third party server 124 associated with a third party in order to match user 102 with the third party. Third party server 124 may be a server associated with a provider and/or service (e.g., ride-sharing service server, taxi, doctor server, housecleaning service server, drycleaner, restaurant, grocery store, utilities, plumber, pharmacy, etc.).

In the exemplary embodiment, caregiver 120 may input registration data (e.g., scheduling/calendar data and task data) into and allow MMM server 110 to access location data relating to caregiver 120, and registration and location data 123 associated with caregiver 120 may be transmitted to MMM server 110 from caregiver 120 (e.g., through mobile device 122 of caregiver 120).

MMM server 110 may further generate arrangements with at least one of caregiver 120 and third party server 124 based upon determined need 118 of user 102 and availability and/or location (relative to a location of user 102) of caregiver 120 (e.g., determined from registration and location data 123 of caregiver 120). For example, if caregiver 120 typically takes user 102 to doctor's appointments, M server 110 may generate arrangements with caregiver 120 when user 102 schedules a doctor's appointment. Further, for example, caregiver 120 may indicate to MMM server 110 (e.g., through registration and location data 123) that caregiver 120 is unavailable on weekdays.

Accordingly, if user 102 makes a doctor's appointment on a weekday, MMM server 110 may generate arrangements with a third party (e.g. a ride-sharing service) via third party server 124 for the third party to take user 102 to doctor's appointment. If the arrangements generated by MMM server 110 with caregiver 120 work for caregiver 120, caregiver 120 may send (e.g., via mobile device 122) a confirmation 127 to MMM server 110. Further, if arrangements generated by MMM server 110 with third party server 124 work for the third party, third party server 124 may send a confirmation 126 to MMM server 110. MMM server 110 may transmit a notification 128 to user 102 including determined need 118 and confirmation 126, 127 from third party server 124 and/or caregiver 120 that determined need 118 will be met by the third party and/or caregiver 120. Further, once generated arrangements are confirmed with caregiver 120 and/or third party server 124 (e.g., through confirmations 126 and/or 127), MMM server may further generate a smart contract (not specifically shown) between user 102 and caregiver 120 and/or the third party, and MMM server may store the generated smart contract on a blockchain (not shown).

In the current embodiment, one caregiver 120 and one third party server 124 are shown for ease of illustration. However, MMM server 110 may transmit determined need 118 to, and make arrangements with, multiple caregivers 120 and/or multiple third party servers 124.

Third party server 124, mobile device 122 of caregiver 120, and/or the blockchain may be capable of communicating with MMM server 110 though an application programming interface (API) 116. In the exemplary embodiment, MMM server 110 may include a firewall (not shown) to protect the private and/or personally identifiable information of user 102. As determined needs 118 and/or arrangements are modified or additional information is received and transmitted, more data may be added to the databases of memory 114 and/or to the blockchain via smart contracts.

In accordance with an exemplary embodiment of MMM server 110, for example, user 102 may be notified on mobile device 106 that user 102 has forgotten to pick up their prescription from a pharmacy for a third time this week. Upon receiving this notification, mobile device 106 may generate user data 108 associated with the notification and transmit generated user data 108 to MMM server 110. From user data 108 and registration data 101, MMM server 110 may determine that a need 118 of user 102 is their prescription and may generate an arrangement to remind user 102 to pick up their prescription or arrange for the prescription to be delivered to user 102. MMM server 110 may communicate with third party server 124 (e.g., associated with the pharmacy of user 102) and determine that the pharmacy offers prescription delivery.

Accordingly, MMM server 110 may arrange the prescription delivery with third party server 124 and receive a delivery confirmation 126 from third party server 124. MMM server 110 may then send notification 128 to user 102 via mobile device 106 (or any other suitable device associated with user 102) notifying user 102 that their prescription will be delivered. Further, MMM server 110 may notify caregiver 120 of user 102 that user 102 had not picked up their prescription from the pharmacy for the third time that week and that MMM server 110 has arranged for the pharmacy to deliver the prescription to user 102. MMM server 110 may prompt caregiver 120 to determine if caregiver 120 wants MMM server 110 to set up an appointment for an evaluation of user 102 with a doctor (via third party server 124) since user 102 forgot to pick up their prescription three times. For example, based upon registration data 101 (e.g., age) and user data 108 (e.g., forgetting prescription), MMM server 110 may determine that user 102 is at risk for dementia and should be further evaluated. Computer-implemented process 100 may include additional, less, or alternate actions, including those discussed elsewhere herein.

### Exemplary Multi-Sided Match Making System

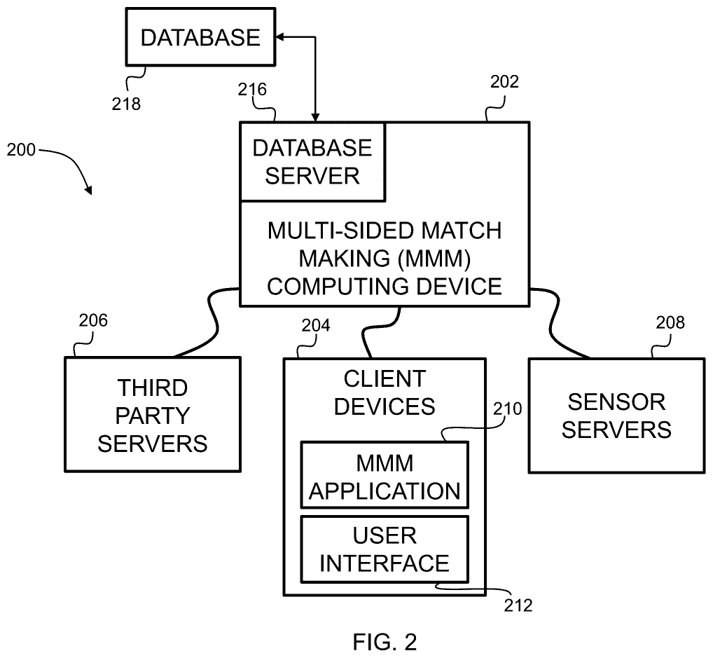

FIG. 2 depicts a view of an exemplary multi-sided match making (“MMM”) system 200 that may be used in implementing process 100 shown in FIG. 1. In the exemplary embodiment, MMM system 200 may be used in matching a user with a provider based upon one or more determined needs of the user. MMM system 200 may include a multi-sided match making (“MMM”) computing device 202. In some embodiments MMM computing device 202 is similar to MMM server 110 (shown in FIG. 1).

In the exemplary embodiment, MMM computing device 202 is in communication with client devices 204, third party servers 206, and sensor servers 208. MMM computing device 202 is also in communication with database 218 and may communicate with a database 218 through a database server 216. In some embodiments, database server 216 is a component of MMM computing device 202. In other embodiments, database server 216 is separate from MMM computing device 202. In some embodiments, MMM system 200 may include a plurality of MMM computing devices 202, client devices 204, third party servers 206, sensor servers 208, and/or databases 218.

In the exemplary embodiment, client devices 204 may be computers that include a web browser or a software application, which enables client devices 204 to access remote computer devices, such as MMM computing device 202, using the Internet or other network. More specifically, client devices 204 may be communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. Client devices 204 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices. Further, MMM computing device 202 may be communicatively coupled to client devices 204 and may receive information from client devices 204.

In the exemplary embodiment, MMM computing device 202 may interact with third party servers 206 associated with third parties that provide a service (e.g., UBER, LYFT, CVS, WALGREENS, etc.). For example, MMM computing device 202 may communicate with an application associated with third party servers 206 to arrange services for user 102 (shown in FIG. 1). MMM computing device 202 and third party servers 206 may be communicatively coupled to one another through the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem.

In the exemplary embodiment, MMM computing device 202 may receive data from sensor servers 208 and may use the data to generate user data 108 (as shown in FIG. 1). For example, MMM computing device 202 may receive user data 108 generated by sensor servers 208 based upon actions 103 (shown in FIG. 1) carried out by user 102. Sensor servers 208 may include smart home device servers (e.g., AMAZON and GOOGLE servers) and wearable device servers (e.g., FITBIT AND APPLE servers). User data 108 may include interaction data from a smart home device (e.g., AMAZON ALEXA and GOOGLE HOME) and activity and/or location data from a wearable device (e.g., FITBIT and APPLE WATCH) and/or autonomous or semi-autonomous vehicles (and/or smart vehicle sensors). MMM computing device 202 and sensor servers 208 may be communicatively coupled through any suitable connection including through the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem.

Database server 216 may be communicatively coupled to database 218 that stores data. In one embodiment, database 218 may include registration data 101 (shown in FIG. 1), user data 108 (shown in FIG. 1), sensor data, mobile device data, confirmation data, and notification data. In the exemplary embodiment, database 218 may be stored remotely from MMM computing device 202. In some embodiments, database 218 may be decentralized. In the exemplary embodiment, a user, such as user 102 or caregiver 120 (shown in FIG. 1), may access database 218 via their respective client devices 204 by logging onto MMM computing device 202, as described herein.

In the exemplary embodiment, client devices 204 include a multi-sided match making (“MEMM”) application 210 and a user interface 212. User interface 212 may be used, for example, to receive notifications from MMM computing device 202 and/or to input and verify information to be sent to MMM computing device 202. MMM application 210 may be, for example, a program or application that runs on client device 204.

In some embodiments, MMM application 210 is accessed remotely by client device 204. MMM application 210 may be hosted by or stored on MMM computing device 202 and accessed by client device 204. For example, MMM application 210 may be stored on and executed by MMM computing device 202. Client device 204 may provide inputs to MMM computing device 202 via a network which are used by MMM computing device 202 to execute MMM application 210. In one embodiment, these inputs may be received by a website hosted by MMM computing device 202. The website may further provide output to client device 204. Client device 204 used by the user (e.g., user 102 and/or caregiver 120) has access to a website (e.g., hosted by MMM computing device 202), application (e.g., MMM application 210), or other tool which the user uses to receive and/or view determined needs 118 (shown in FIG. 1), third party confirmation 126 (shown in FIG. 1), caregiver confirmations 127 (shown in FIG. 1), notifications 128 (shown in FIG. 1), and analyzed user data 108 (shown in FIG. 1), provided by MMM computing device 202 to the user. MMM system may be configured with additional, less, or alternate functionality, including that discussed elsewhere herein.

### Exemplary User Computer Device

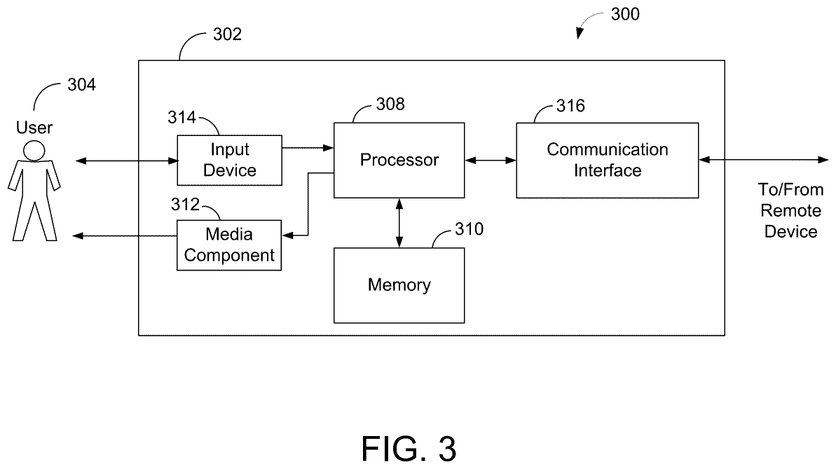

FIG. 3 illustrates an exemplary configuration 300 of an exemplary user computer device 302. In some embodiments, user computing device 302 may be in communication with a multi-sided match making computing device (such as MMM server 110, shown in FIG. 1 or MMM computing device 202, shown in FIG. 2). User computing device 302 may be representative of, but is not limited to client devices 204, third party servers 206, and/or sensor servers 208. For example, user computing device 302 may be a mobile device, smartphone, tablet, smartwatch, wearable electronic, laptop, desktop, or another type of computing device associated with an account holder (e.g., user 102 and/or caregiver 120).

User computer device 302 may be operated by a user 304 (e.g., a user of MMM system 200, shown in FIG. 2 and substantially similar to user 102 and caregiver 120, shown in FIG. 1). User computer device 302 may receive input from user 304 via an input device 314. User computer device 302 includes a processor 308 for executing instructions. In some embodiments, executable instructions may be stored in a memory area 310. Processor 308 may include one or more processing units (e.g., in a multi-core configuration), and processor 308 may include, be in communication with, and/or be associated with one or more transceivers (not specifically shown). Memory area 310 may be any device allowing information such as executable instructions and/or user and registration data to be stored and retrieved. Memory area 310 may include one or more computer-readable media.

User computer device 302 also may include at least one media output component 312 for presenting information to user 304. Media output component 312 may be any component capable of conveying information to user 304 and may be used to at least partially implement user interface 212 (shown in FIG. 2). In some embodiments, media output component 312 may include an output adapter (not shown), such as a video adapter and/or an audio adapter. An output adapter may be operatively coupled to processor 308 and operatively coupleable to an output device, such as a display device (e.g., a cathode ray tube (CRT), liquid crystal display (LCD), light emitting diode (LED) display, or “electronic ink” display) or an audio output device (e.g., a speaker or headphones).

In some embodiments, media output component 312 may be configured to present a graphical user interface (e.g., a web browser and/or a client application) to user 304. A graphical user interface may include, for example, calendars for user 102 and/or caregiver 120, notifications for user 102, determined needs 118 of user 102, and/or an activity profile of user 102.

In some embodiments, user computer device 302 may include input device 314 for receiving input from user 304. User 304 may use input device 314 to, without limitation, interact with MMM system 200 (e.g., using MMM application 210), MMM computing device 202, or any of client devices 204, third party servers 206, and sensor servers 208 (shown in FIG. 2). Input device 314 may include, for example, a keyboard, a pointing device, a mouse, a stylus, and/or a touch sensitive panel (e.g., a touch pad or a touch screen) and may be used to at least partially implement user interface 212 (shown in FIG. 2). A single component, such as a touch screen, may function as both an output device of media output component 312 and input device 314. User computer device 302 may further include at least one sensor, including, for example, a gyroscope, an accelerometer, a position detector, a biometric input device, and/or an audio input device. In some embodiments, at least some data collected by user computer device 302 may be transmitted to MMM computing device 202. In the exemplary embodiment, data collected by user computer device 302 may be included in user data 108.

User computer device 302 may also include a communication interface 316, communicatively coupled to any of MMM computing device 202, client devices 204, third party servers 206, and sensor servers 208. Communication interface 316 may include, for example, a wired or wireless network adapter and/or a wireless data transceiver for use with a mobile telecommunications network.

Stored in memory area 310 may be, for example, computer-readable instructions for providing a user interface to user 304 via media output component 312 and, optionally, receiving and processing input from input device 314. The user interface may include, among other possibilities, a web browser and/or a client application. Web browsers enable users, such as user 304, to display and interact with media and other information typically embedded on a web page or a website hosted by MMM computing device 202 and/or client device 204. A client application may allow user 304 to interact with, for example, any of MMM computing device 202, client devices 204, third party servers 206, and sensor servers 208. For example, instructions may be stored by a cloud service and the output of the execution of the instructions sent to the media output component 312. User computing device 302 may include additional, less, or alternate functionality, including that discussed elsewhere herein.

### Exemplary Server Device

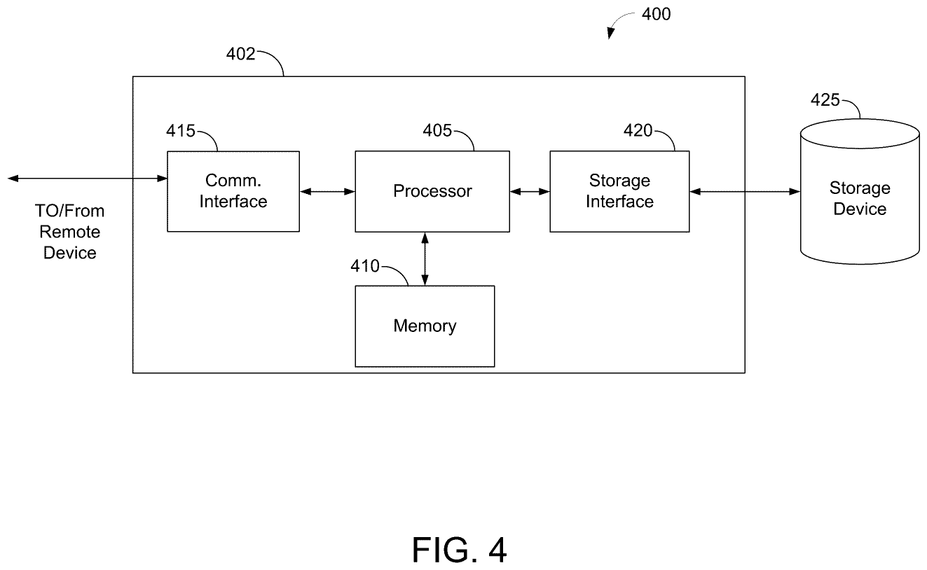

FIG. 4 depicts an exemplary configuration 400 of an exemplary server computer device 402, in accordance with one embodiment of the present disclosure. Server computer device 402 may include, but is not limited to, MMM computing device 202 (shown in FIG. 2). Server computer device 402 may include a processor 405 for executing instructions. Instructions may be stored in a memory area 410. Processor 405 may include one or more processing units (e.g., in a multi-core configuration), and processor 405 may include, be in communication with, and/or be associated with one or more transceivers (not specifically shown).

Processor 405 may be operatively coupled to a communication interface 415 such that server computer device 402 may be capable of communicating with a remote device such as another server computer device 402 or a user computing device, such as client device 204 (shown in FIG. 2). For example, communication interface 415 may receive requests from or transmit requests to client devices 204 via the Internet.

Processor 405 may also be operatively coupled to a storage device 425. Storage device 425 may be any computer-operated hardware suitable for storing and/or retrieving data, such as, but not limited to, data associated with database 218 (shown in FIG. 2). In some embodiments, storage device 425 may be integrated in server computer device 402. For example, server computer device 402 may include one or more hard disk drives as storage device 425. In other embodiments, storage device 425 may be external to server computer device 402 and may be accessed by a plurality of server computer devices 402. For example, storage device 425 may include a storage area network (SAN), a network attached storage (NAS) system, and/or multiple storage units such as hard disks and/or solid state disks in a redundant array of inexpensive disks (RAID) configuration.

In some embodiments, processor 405 may be operatively coupled to storage device 425 via a storage interface 420. Storage interface 420 may be any component capable of providing processor 405 with access to storage device 425. Storage interface 420 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 405 with access to storage device 425.

Processor 405 executes computer-executable instructions for implementing aspects of the disclosure. In some embodiments, processor 405 may be transformed into a special purpose microprocessor by executing computer-executable instructions or by otherwise being programmed.

### Exemplary Computer-Implemented Method

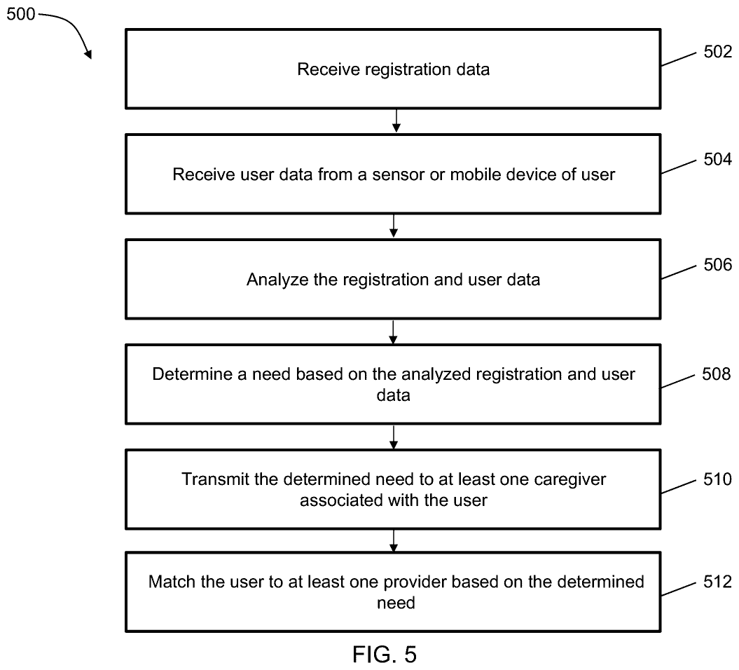

FIG. 5 depicts a flow chart illustrating a computer-implemented method 500 for matching users to providers based upon a determined need. In the exemplary embodiment, method 500 may be implemented by a multi-sided match making computer system such as MMM server 110 (shown in FIG. 1) and MMM computing device 202 (shown in FIG. 2).

Method 500 may include receiving 502 registration data from a user (e.g., user 102 shown in FIG. 1). Method 500 may also include receiving 504 user data (e.g., user data 108 shown in FIG. 1) from at least one of a sensor (e.g., sensors 104 shown in FIG. 1) or mobile device (e.g., mobile device 106 shown in FIG. 1) of the user. The user data may be generated in response to an action carried out by the user.

Method 500 may further include analyzing 506 the received 502, 504 registration and user data and determining 508 a need (e.g., determined need 118 shown in FIG. 1) of the user based upon the analyzed 506 registration and user data. Method 500 may include transmitting 510 the determined need to at least one caregiver (e.g., caregivers 120 shown in FIG. 1) associated with the user. Method 500 may additionally include matching 512 the user to at least one provider (e.g., a third party associated with third party server 124 and/or caregiver 120 shown in FIG. 1) based upon the determined 508 need. Matching 512 may occur between the user and the provider because the provider may offer to take care of the determined 508 need. Method 500 may include additional, less, or alternate actions, including those discussed elsewhere herein.

### Exemplary Computer Device

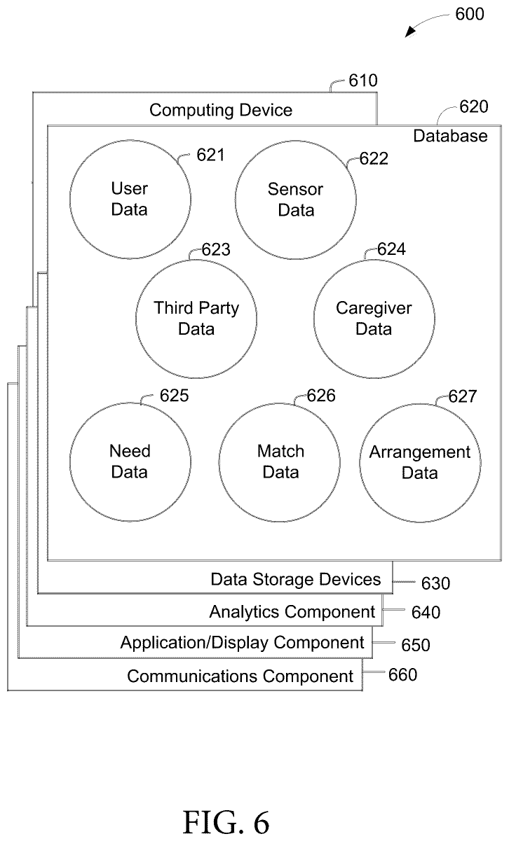

FIG. 6 depicts a diagram 600 of components of one or more exemplary computing devices 610 that may be used in multi-sided match making system 200 (shown in FIG. 2). In some embodiments, computing device 610 may be similar to MMM server 110 (shown in FIG. 1) and MMM computing device 202 (shown in FIG. 2). Database 620 may be coupled with several separate components within computing device 610, which perform specific tasks. In this embodiment, database 620 may include user data 621, sensor data 622, third party data 623, caregiver data 624, need data 625, match data 626, and arrangement data 627. In some embodiments, database 620 is similar to database 218 (shown in FIG. 2). In further embodiments, match data 626 and/or arrangement data 627 may be stored as smart contracts on a blockchain that is in communication with database 620.

Computing device 610 may include database 620, as well as data storage devices 630. Computing device 610 may also include an analytics component 640 for analyzing registration data 101 and user data 108 and determining a need 118 (all shown in FIG. 1). Computing device 610 may further include application/display component 650 for generating and displaying information to users, such as through MMM application 210 (shown in FIG. 2), and supporting MMM application 210. Moreover, computing device 610 may include communications component 660 for receiving and transmitting data, such as user data 621, sensor data 622, third party data 623, caregiver data 624, need data 625, match data 626, and arrangement data 627. Computing devices 610 may include additional, less, or alternate functionality, including that discussed elsewhere herein.

### Exemplary Multi-Sided Caregiver Platform Functionality

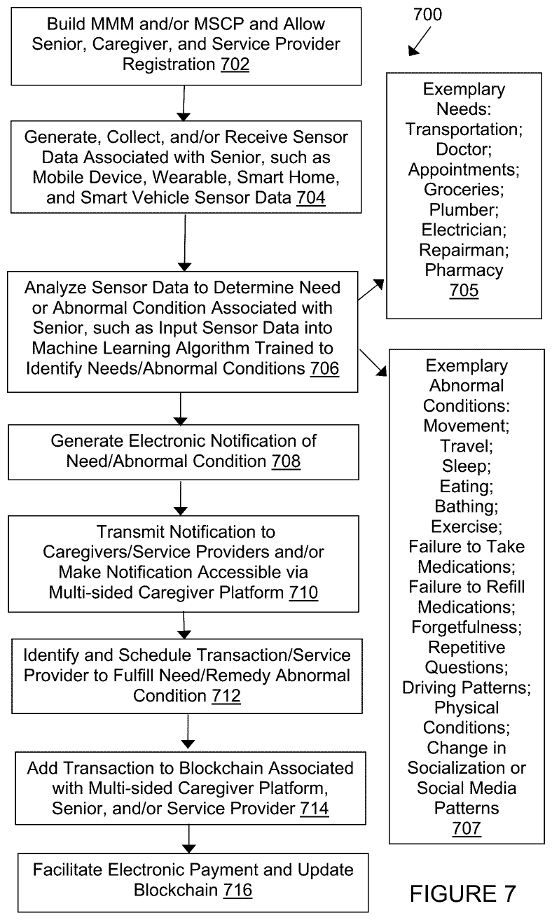

FIG. 7 illustrates an exemplary computer-implemented method 700 associated with a multi-sided caregiver platform (“MSCP”) computer system and/or multi-sided match making (“MMM”) computer system, which are together referred to as “platforms.” Computer-implemented method 700 may be substantially similar to process 100 (shown in FIG. 1) and/or method 500 (shown in FIG. 5). Computer-implemented method 700 may include building 702 the platforms. The platforms may be configured to allow 702 seniors, senior patients, caregivers (such as family members and professional caregivers), and service and product providers to register (e.g., with registration data 101 of user 102 and/or registration and location data 123 of caregiver 120, shown in FIG. 1) with the MMM and/or MSCP computer systems, such as build a profile and enter name, address, and preference information.

In the exemplary embodiment, computer-implemented method 700 may include, via one or more processors and/or associated transceivers, receiving, generating, and/or collecting 704 sensor data and/or mobile device data (e.g., user data 108 generated by sensors 104 and/or mobile device 106, shown in FIG. 1) associated with a senior. For instance, computer-implemented method 700 may include receiving 704, via one or more processors and/or associated transceivers and via wireless communication and/or data transmission over one or more radio frequency links, sensor and/or mobile device data (e.g., user data 108, shown in FIG. 1) associated with a user or customer, such as a senior or senior patient. The sensor and/or mobile device data may be generated and transmitted by mobile devices, smart home sensors and controllers, autonomous or semi-autonomous or smart vehicle controllers or processors, wearable devices, smart aerial devices or smart drones, smart infrastructure sensors, etc.

Computer-implemented method 700 may include, via one or more processors and/or associated transceivers, analyzing 706 the sensor data to determine or identify a developing or unmet need (e.g., determined need 118, shown in FIG. 1) of the senior, or determine or identify an abnormal condition associated with the senior. In some embodiments, the sensor data may be analyzed 706 by being input into a machine learning algorithm, model, or program trained to identify developing or unmet needs 705 of a senior, and/or determine or identify an abnormal condition 707 associated with the senior. In some embodiments, unmet needs 705 and abnormal conditions 707 may be substantially similar to determined need 118 (shown in FIG. 1).

In some embodiments, developing or unmet need 705 of the senior identified by the computer system may include a need for: transportation, a doctor, various appointments, groceries, plumbing services, electrical services, various repair work, pharmacy attention, and/or other unmet needs 705, including those discussed elsewhere herein.

In some embodiments, abnormal conditions 707 associated with the senior identified by the computer system may include abnormal, or a change in: (a) movement, travel, sleep, eating, bathing, showering, exercise, and/or socialization patterns; (b) forgetfulness; (c) physical conditions; (d) socialization, social media usage, mobile device usage, telephone usage, email or text communication; and/or (e) other behavior patterns discussed herein. Abnormal conditions 707 associated with the senior identified by the computer system may include failure to take medications; failure to refill medications; change or lapse in memory; repetitive questions or speech; and/or other conditions, including those discussed elsewhere herein.

Computer-implemented method 700 may include generating 708, via one or more processors and/or associated transceivers, an electronic notification of an identified need or abnormal condition of the senior, such as those described elsewhere herein. Computer-implemented method 700 may include, via one or more processors and/or associated transceivers, transmitting 710, via wireless communication or data transmission over one or more radio frequency links, the generated 708 electronic notification to a mobile device (e.g., mobile device 122, shown in FIG. 1), or other computing device, associated with family members and other caregivers (e.g., caregiver 120, shown in FIG. 1), and/or service providers (e.g., third parties, described with respect to FIG. 1), and otherwise make 710 the generated 708 electronic notification available to the network participants via the platforms.

Computer-implemented method 700 may include, via one or more processors and/or associated transceivers, identifying and/or scheduling 712 a transaction and/or service provider to meet unmet need 705 and/or remedy abnormal condition 707 (e.g., making arrangements with the provider, as described herein). The service provider may identified based upon social media reviews and/or proximity to the customer/senior. For instance, service providers may be selected based upon proximity to a senior's mobile device GPS (Global Positioning System) location and availability, as well as each service provider's location, proximity to the senior, and availability.

Computer-implemented method 700 may include, via one or more processors and/or associated transceivers, adding 714 each transaction to a blockchain, such as a blockchain associated with the platforms or the senior, caregiver, or service provider. Each transaction may also be also associated with a smart contract.

Computer-implemented method 700 may include, via one or more processors and/or associated transceivers, transferring 716 funds electronically from a virtual account associated with the senior to a financial account of the service provider. For instance, payment for each transaction (e.g., arrangement and/or match) may be electronic in some embodiments, and may be recorded on the blockchain.

### EXEMPLARY EMBODIMENTS

In one aspect, a multi-sided match making (“MMM”) computer system for matching consumers to providers may be provided. The MMM computer system may include at least one processor in communication with at least one memory device, the at least one processor is programmed to: (1) receive registration data from a user; (2) receive user data from at least one of a sensor and a mobile device associated with the user, wherein the user data is generated by the at least one of the sensor and the mobile device in response to an action carried out by the user; (3) analyze the registration data and the user data; (4) determine a need based upon the analyzed registration and user data; (5) transmit the determined need to at least one caregiver associated with the user; and/or (6) match the user to at least one provider based upon the determined need, wherein the provider is at least one of the caregiver, another caregiver, and a service, and wherein the provider is able to meet the determined need for the user. The MMM computer system may include additional, less, or alternate functionality, including that discussed elsewhere herein.

For instance, the processor may be further programmed to: (i) automatically generate arrangements with the provider based upon the determined need of the user; (ii) transmit the generated arrangements to the provider; (iii) receive confirmation from the provider regarding the arrangements, wherein the confirmation confirms that the provider will meet the determined need of the user; and/or (iv) send a notification to the user including the confirmed arrangements.

The processor may be programmed to receive feedback from the user regarding the generated arrangements. The processor may be further programmed to store in the at least one memory device information including (i) the received user and registration data, (ii) the determined need of the user, (iii) the matching of the user and the provider for the determined need of the user, (iv) the generated arrangements, and/or (v) the feedback from the user regarding the generated arrangements.

The processor may be programmed to determine needs for the user, match the user to the provider, and generate arrangements for the user by using machine learning techniques on the stored information. The processor may be further programmed to: discover patterns of the user from the stored information; and/or recognize a pattern disruption of the user, wherein the pattern disruption is recognized when the user data demonstrates a deviation from the discovered patterns of the user.

The registration data may include at least one of an age, a birthdate, a height, a weight, a medical history, and preferred doctors and pharmacies of the user. The sensors may include mobile devices, smart home devices and wearable devices, and/or other sensors, such as autonomous or semi-autonomous vehicle sensors, or aerial device sensors. The action carried out by the user may include at least one of interacting with the smart home device of the user, autonomous or semi-autonomous vehicle of the user, scheduling an appointment on the mobile device, receiving a notification on the mobile device, conversing on the mobile device, and exercising.

In another aspect, a computer-implemented method for matching consumers to providers may be provided. The method may be implemented using a multi-sided match making (“MMM”) computer system including at least one processor in communication with at least one memory device. The method may include: (1) receiving registration data from a user; (2) receiving user data from at least one of a sensor and a mobile device associated with the user, wherein the user data is generated by the at least one of the sensor and the mobile device in response to an action carried out by the user; (3) analyzing the registration data and the user data; (4) determining a need based upon the analyzed registration and user data; (5) transmitting the determined need to at least one caregiver associated with the user; and/or (6) matching the user to at least one provider based upon the determined need, wherein the provider is at least one of the caregiver, another caregiver, and a service, and wherein the provider is able to meet the determined need for the user. The method may include additional, less, or alternate functionality, including that discussed elsewhere herein.

For instance, the method may further include (i) automatically generating arrangements with the provider based upon the determined need of the user; (ii) transmitting the generated arrangements to the provider; (iii) receiving confirmation from the provider regarding the arrangements, wherein the confirmation confirms that the provider will meet the determined need of the user; and/or (iv) sending a notification to the user including the confirmed arrangements.

The method may include receiving feedback from the user regarding the generated arrangements. The method may include storing in the at least one memory device information including (i) the received user and registration data, (ii) the determined need of the user, (iii) the matching of the user and the provider for the determined need of the user, (iv) the generated arrangements, and/or (v) the feedback from the user regarding the generated arrangements.

The method may include determining needs for the user, matching the user to the provider, and generating arrangements for the user by using machine learning techniques on the stored information. The method may include: discovering patterns of the user from the stored information; and/or recognizing a pattern disruption of the user, wherein the pattern disruption is recognized when the user data demonstrates a deviation from the discovered patterns of the user.

The registration data may include at least one of an age, a birthdate, a height, a weight, a medical history, preferred doctors and pharmacies of the user, preferred transportation companies of the user, and other preferred service providers of the user. The sensors include smart home devices and wearable devices, and the action carried out by the user includes at least one of interacting with the smart home device of the user, scheduling an appointment on the mobile device, receiving a notification on the mobile device, conversing on the mobile device, and exercising. In other embodiments, the sensors may also include autonomous or semi-autonomous vehicle sensors, and/or other sensors, such as smart city sensors or aerial device sensors.

In another aspect, at least one non-transitory computer-readable media having computer-executable instructions thereon is provided. Wherein when executed by at least one processor of a multi-sided match making (“MMM”) computer system, the instructions cause the at least one processor of the MMM computer system to: (1) receive registration data from a user; (2) receive user data from at least one of a sensor and a mobile device associated with the user, wherein the user data is generated by the at least one of the sensor and the mobile device in response to an action carried out by the user; (3) analyze the registration data and the user data; (4) determine a need based upon the analyzed registration and user data; (5) transmit the determined need to at least one caregiver associated with the user; and/or (6) match the user to at least one provider based upon the determined need, wherein the provider is at least one of the caregiver, another caregiver, and a service, and wherein the provider is able to meet the determined need for the user.

The instructions may further cause the at least one processor of the MMM computing device to: automatically generate arrangements with the provider based upon the determined need of the user; transmit the generated arrangements to the provider; receive confirmation from the provider regarding the arrangements, wherein the confirmation confirms that the provider will meet the determined need of the user (and/or abnormal condition associated with the user, as discussed elsewhere herein); and/or send a notification to the user including the confirmed arrangements.

The instructions may also cause the at least one processor of the MMM computing device to: receive feedback from the user regarding the generated arrangements. The instructions may further cause the at least one processor of the MMM computing device to: store in the at least one memory device information including (i) the received user and registration data, (ii) the determined need of the user, (iii) the matching of the user and the provider for the determined need of the user (and/or abnormal condition associated with the user), (iv) the generated arrangements, and/or (v) the feedback from the user regarding the generated arrangements.

The instructions may cause the at least one processor of the MMM computing device to: determine needs for the user, match the user to the provider, and generate arrangements for the user by using machine learning techniques on the stored information. The instructions may further cause the at least on processor of the MMM computing device to: discover patterns of the user from the stored information; and/or recognize a pattern disruption of the user, wherein the pattern disruption is recognized when the user data demonstrates a deviation from the discovered patterns of the user. The instructions may direct additional, less, or alternate functionality, including that discussed elsewhere herein.

### Exemplary MSCP Embodiments

In one aspect, a multi-sided caregiver platform (MSCP) computer system for matching consumers to providers may be provided. The MSCP computer system may include at least one processor and associated transceiver in communication with at least one memory device. The at least one processor and/or transceiver may be programmed to: (i) allow participants to register with the MSCP, such as via wireless communication or data transmission and a dedicated website and/or mobile application, the participants including seniors, caregivers, and/or service providers; (ii) receive sensor data associated with a senior via wireless communication or data transmission from a transceiver associated with a mobile device, smart home controller, smart vehicle, or wearable device, the sensor data including mobile device sensor data, smart home sensor data, smart vehicle sensor data, wearable sensor data, or other sensor data associated with the senior; (iii) analyze the sensor data to determine or identify a need or abnormal condition associated with the senior; (iv) generate an electronic notification of, or detailing, the need or abnormal condition associated with the senior; and/or (v) transmit the electronic notification, via wireless communication or data transmission to a mobile device or other computing device of one or more caregivers and/or service providers, and/or otherwise make the electronic notification accessible via the dedicated website and/or mobile application to facilitate addressing the need and/or abnormal condition associated with the senior.

In another aspect, a multi-sided caregiver platform (MSCP) computer system for matching consumers to providers may be provided. The MSCP computer system may include at least one processor and associated transceiver in communication with at least one memory device. The at least one processor and/or associated transceiver may be programmed to: (i) allow participants to register with the MSCP, such as via wireless communication or data transmission and a dedicated website and/or mobile application, the participants including seniors, caregivers, and/or service providers; (ii) generate or collect sensor data associated with a senior, the sensor data including mobile device sensor data, smart home sensor data, smart vehicle sensor data, wearable sensor data, or other sensor data associated with the senior; (iii) analyze the sensor data to determine or identify a need or abnormal condition associated with the senior; (iv) generate an electronic notification of, or detailing, the need or abnormal condition associated with the senior; and/or (v) transmit the electronic notification, via wireless communication or data transmission to a mobile device or other computing device of one or more caregivers and/or service providers, and/or otherwise make the electronic notification accessible via the dedicated website and/or mobile application to facilitate addressing the need and/or abnormal condition associated with the senior.

The foregoing MSCP computer systems may include additional, less, or alternate functionality, including that discussed elsewhere herein. For instance, to analyze the sensor data to determine or identify a need or abnormal condition associated with the senior, the at least one processor may be configured to: input the sensor data into a machine learning algorithm, model, or program trained to identify needs and/or abnormal conditions from sensor data, including mobile device, smart home, smart vehicle, and/or wearable device sensor data.

A need associated with the senior identified may be a need for transportation services, such as a taxi or transportation company; medical or doctor services; repair services; electrician or plumbing services; pharmaceutical services; grocery delivery services; and/or scheduling an appointment.

An abnormal condition associated with the senior identified may be abnormal, and/or a change in, movement of the senior within a home; movement of the senior outside of a home (i.e., movement about or around town); and/or travel activity of the senior.

An abnormal condition associated with the senior identified may be abnormal, and/or a change in, sleep, or an abnormal, and/or a change in, sleep pattern or routine of the senior. An abnormal condition associated with the senior identified may be abnormal, and/or a change in, eating, or an abnormal, and/or a change in, eating pattern or routine of the senior. An abnormal condition associated with the senior identified may be abnormal, and/or a change in, bathing or showering, or an abnormal, and/or a change in, bathing or showering pattern or routine of the senior.

An abnormal condition associated with the senior identified may be abnormal, and/or a change in, exercise, or an abnormal, and/or a change in, exercise pattern or routine of the senior. An abnormal condition associated with the senior identified may be abnormal, and/or a change in, gait of the senior. An abnormal condition associated with the senior identified may be abnormal, and/or a change in, appearance of the senior.

An abnormal condition associated with the senior identified may be failure to take prescribed medications on time; and/or failure to refill prescribed medications on time.

An abnormal condition associated with the senior identified may be forgetfulness or the onset of forgetfulness; repetitive questions; and/or repetitive speech.

The sensor data may include vehicle telematics data (e.g., braking, cornering, speed, location, acceleration data collected from a smart vehicle or a mobile device), and an abnormal condition associated with the senior identified may be abnormal (or a change in) or risky driving patterns.

The vehicle telematics data or vehicle data may also include vehicle maintenance data, and the need identified may include a need for vehicle maintenance. The MSCP computer system may identify a preferred or recommended car dealership or body shop that is qualified to perform the maintenance, and schedule an appointment to perform the vehicle maintenance, and generate associated electronic messages.

The home sensor data and/or home telematics data may include electricity, energy, fuel, gas, or water usage of the home. The home sensor data and/or home telematics data may also include home maintenance data, and the need identified may include a need for home maintenance. The MSCP computer system may identify a preferred or recommended repairman, electrician, plumber, etc. that is qualified to perform the maintenance, and schedule an appointment to perform the home maintenance, and generate associated electronic messages.

An abnormal condition associated with the senior identified may be an abnormal, and/or a change in, physical condition (e.g., heart rate). An abnormal condition associated with the senior identified may be abnormal, and/or a change in, socialization; social media usage; email or text usage; responsiveness to telephone calls, emails, texts, or other electronic communications; responsive to social media communications; and/or telephone usage.

The MSCP computer system may be further configured to identify and/or schedule a transaction and/or service provider to fulfill the need identified and/or remedy the abnormal condition identified; add the transaction identified to a blockchain associated with the MSCP, senior, and/or one or more caregivers and/or service providers; and/or schedule an appointment with a service provider to fulfill the need identified, and/or remedy the abnormal condition identified.

In another aspect, a computer-implemented method for matching consumers to providers may be provided. The method may be implemented using a multi-sided caregiver platform (MSCP) computer system including at least one processor and associated transceiver in communication with at least one memory device. The method, via the at least one processor and/or associated transceiver, may include (1) allowing participants to register with the MSCP, such as via wireless communication or data transmission and a dedicated website and/or mobile application, the participants including seniors, caregivers, and/or service providers; (2) receiving sensor data associated with a senior via wireless communication or data transmission from a transceiver associated with a mobile device, smart home controller, smart vehicle, or wearable device, the sensor data including mobile device sensor data, smart home sensor data, smart vehicle sensor data, wearable sensor data, or other sensor data associated with the senior; (3) analyzing the sensor data to determine or identify a need or abnormal condition associated with the senior; (4) generating an electronic notification of, or detailing, the need or abnormal condition associated with the senior; and/or (5) transmitting the electronic notification, via wireless communication or data transmission to a mobile device or other computing device of one or more caregivers and/or service providers, and/or otherwise making the electronic notification accessible via the dedicated website and/or mobile application to facilitate addressing the need and/or abnormal condition associated with the senior.

In another aspect, a computer-implemented method for matching consumers to providers may be provided. The method may be implemented using a multi-sided caregiver platform (MSCP) computer system may include at least one processor and associated transceiver in communication with at least one memory device. The method, via the at least one processor and/or associated transceiver, may include: (1) allowing participants to register with the MSCP, such as via wireless communication or data transmission and a dedicated website and/or mobile application, the participants including seniors, caregivers, and/or service providers; (2) generating or collecting sensor data associated with a senior, the sensor data including mobile device sensor data, smart home sensor data, smart vehicle sensor data, wearable sensor data, or other sensor data associated with the senior; (3) analyzing the sensor data to determine or identify a need or abnormal condition associated with the senior; (4) generating an electronic notification of, or detailing, the need or abnormal condition associated with the senior; and/or (5) transmitting the electronic notification, via wireless communication or data transmission to a mobile device or other computing device of one or more caregivers and/or service providers, and/or otherwise making the electronic notification accessible via the dedicated website and/or mobile application to facilitate addressing the need and/or abnormal condition associated with the senior. The foregoing computer-implemented methods may include additional, less, or alternate actions, including those discussed elsewhere herein and with respect to the MSCP computer system.

### Machine Learning & Other Matters

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on mobile computing devices, or associated with smart infrastructure or remote servers), and/or via computer executable instructions stored on non-transitory computer-readable media or medium.

In some embodiments, a multi-sided match making computing device is configured to implement machine learning, such that the multi-sided match making computing device “learns” to analyze, organize, and/or process data without being explicitly programmed. Machine learning may be implemented through machine learning methods and algorithms (“ML methods and algorithms”). In an exemplary embodiment, a machine learning module (“ML module”) is configured to implement ML methods and algorithms. In some embodiments, ML methods and algorithms are applied to data inputs and generate machine learning outputs (“NIL outputs”). Data inputs may include but are not limited to: user data, sensor data, third party data, caregiver data, need data, match data, home telematics data, vehicle telematics data, smart home and smart vehicle data, and/or arrangement data. ML outputs may include but are not limited to: user data, need data, match data, and/or arrangement data. In some embodiments, data inputs may include certain ML outputs.

In some embodiments, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of machine learning, such as supervised learning, unsupervised learning, and reinforcement learning.

In one embodiment, the ML module employs supervised learning, which involves identifying patterns in existing data to make predictions about subsequently received data. Specifically, the ML module is “trained” using training data, which includes example inputs and associated example outputs. Based upon the training data, the ML module may generate a predictive function which maps outputs to inputs and may utilize the predictive function to generate ML outputs based upon data inputs. The example inputs and example outputs of the training data may include any of the data inputs or ML outputs described above. For example, a ML module may receive training data comprising user data, sensor data, and match data associated with the user data and match data. The ML module may then generate a model which maps match data to aspects of user data and sensor data. The ML module may then generate match data as a ML output based upon subsequently received user data and sensor data.

In another embodiment, an ML module may employ unsupervised learning, which involves finding meaningful relationships in unorganized data. Unlike supervised learning, unsupervised learning does not involve user-initiated training based upon example inputs with associated outputs. Rather, in unsupervised learning, the ML module may organize unlabeled data according to a relationship determined by at least one ML method/algorithm employed by the ML module. Unorganized data may include any combination of data inputs and/or ML outputs as described above. For example, a ML module may receive unlabeled data comprising user data, sensor data, and need data. The ML module may employ an unsupervised learning method such as “clustering” to identify patterns and organize the unlabeled data into meaningful groups. The newly organized data may be used, for example, to generate a model which associates user data and sensor data to determined needs.

In yet another embodiment, a ML module may employ reinforcement learning, which involves optimizing outputs based upon feedback from a reward signal. Specifically, the ML module may receive a user-defined reward signal definition, receive a data input, utilize a decision-making model to generate a ML output based upon the data input, receive a reward signal based upon the reward signal definition and the ML output, and alter the decision-making model so as to receive a stronger reward signal for subsequently generated ML outputs. Other types of machine learning may also be employed, including deep or combined learning techniques.

The reward signal definition may be based upon any of the data inputs or ML outputs described above. For example, a ML module may implement reinforcement learning in generating match data for users. The ML module may utilize a decision-making model to generate match data for users based upon sensor data, and may further receive user-satisfaction data indicating a level of satisfaction experienced by a user and a caregiver who engaged in a transaction. A reward signal may be generated by comparing the user-satisfaction data to the sharing score between the user and the caregiver.

Based upon the reward signal, the ML module may update the decision-making model such that subsequently generated sharing scores more accurately predict user satisfaction. For example, the ML module may determine that a specific driver from a third-party ride sharing service has driven a user to their last four doctor's appointments. The user may have rated the specific driver very highly after each ride. Accordingly, the ML module may learn to automatically book the specific driver for the user when it is determined that the user needs a ride.

### Blockchain Functionality

The systems and methods described herein, in some embodiments, relate to, a multi-sided caregiver platform that is managed by using one or more blockchains. In one exemplary embodiment, the process may be performed by a blockchain-based multi-sided caregiver platform computing device. Blockchain functionality may be used to log interactions, transactions, and/or payments between seniors, caregivers, and/or service providers.

A blockchain is a distributed database that maintains a continuously-growing list of ordered records, known as blocks. Each block may contain at least a timestamp and a link to the previous block in the chain. The link to the previous block may be a hash of the previous block. For example, in the case of a smart contract, the first block may contain the initial contract between a senior and a caregiver or service provider. The second block may contain a modification to the smart contract that was requested by the senior and approved by the caregiver or service provider. The second block may contain a hashed copy of the first block as well. The third block may contain one or more additional terms for the smart contract and a hashed copy of the second block. This continues on with each block adding on to the next while containing a hash of the previous blocks in the blockchain.

To ensure the security of the information contained in the blockchain, copies of the blockchain are distributed across multiple computer devices, known as nodes. These nodes maintain the blockchain, update the blockchain when changes occur, and ensure the stability of the blockchain itself. In some embodiments, nodes may also be used to calculate the hash of the previous blocks. As the blockchain grows, the processing power needed to calculate the hash of the previous blocks grows as well. In these embodiments, the processing of the hash may be distributed over multiple computer devices to improve the speed of processing and/or to not overburden the hashing processor. When a node processes (hashes) a block, that node is known as a miner, where the action of validating and hashing the block is also known as mining.

### Additional Considerations

With the foregoing, users and caregivers may opt-in or register to a multi-sided match making program or other type of program. After the users and caregivers give their affirmative consent or permission, a multi-sided match making remote server may collect data from the mobile devices, user computing devices, smart home controllers, smart vehicles, autonomous or semi-autonomous vehicles, smart infrastructure, smart buildings, smart aerial devices (e.g., drones), and/or other smart devices, such as with the permission or affirmative consent of the users and caregivers. The data collected may be related to user activities and/or user/caregiver schedules and current locations.

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one embodiment, a computer program is provided, and the program is embodied on a computer readable medium. In an exemplary embodiment, the system is executed on a single computer system, without requiring a connection to a server computer. In a further embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

In some embodiments, registration of users for the multi-sided match making platform includes opt-in informed consent of users to data usage by the smart home devices, wearable devices, mobile devices, autonomous vehicles, and/or smart vehicles consistent with consumer protection laws and privacy regulations. In some embodiments, the registration data, the user data, and/or other collected data may be anonymized and/or aggregated prior to receipt such that no personally identifiable information (PII) is received. In other embodiments, the system may be configured to receive registration data and/or other collected data that is not yet anonymized and/or aggregated, and thus may be configured to anonymize and aggregate the data. In such embodiments, any PII received by the system is received and processed in an encrypted format, or is received with the consent of the individual with which the PII is associated. In situations in which the systems discussed herein collect personal information about individuals, or may make use of such personal information, the individuals may be provided with an opportunity to control whether such information is collected or to control whether and/or how such information is used. In addition, certain data may be processed in one or more ways before it is stored or used, so that personally identifiable information is removed.

As used herein, an element or step recited in the singular and proceeded with the word “a” or “an” should be understood as not excluding plural elements or steps, unless such exclusion is explicitly recited. Furthermore, references to “exemplary embodiment” or “one embodiment” of the present disclosure are not intended to be interpreted as excluding the existence of additional embodiments that also incorporate the recited features.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A computer system for matching consumers to providers, the computer system comprising at least one processor in communication with at least one memory device and one or more sensors associated with a vehicle of a user, the at least one processor configured to:
generate a plurality of driving patterns of behavior of the user from historical vehicle telematics data collected by the one or more sensors including a good driving pattern for the user;
in response to receiving current vehicle telematics data associated with the user, detect one or more pattern disruptions of the plurality of driving patterns of behavior of the user;
determine a need of the user based upon the one or more pattern disruptions, the determined need addressable by one or more caregivers associated with the user;
in response to determining that none of the one or more caregivers is available to address the determined need of the user, determine a third-party service provider that is able to meet the determined need for the user;
automatically generate arrangements with the determined third-party service provider; and
transmit the generated arrangements to a computer device associated with the determined third-party service provider.

2. The computer system of claim 1, wherein the at least one processor is further configured to:
receive registration data from the user; and
generate the plurality of driving patterns of behavior of the user by analyzing the registration data.

3. The computer system of claim 1, wherein the at least one processor is further configured to continuously receive user data from the one or more sensors, wherein the user data includes the historical vehicle telematics data, and wherein the historical vehicle telematics data includes movement data.

4. The computer system of claim 1, wherein the one or more pattern disruptions include a negative driving pattern for the user.

5. The computer system of claim 1, wherein the at least one processor is further configured to:
receive schedule information from the one or more caregivers; and
determine availability of the one or more caregivers based on the received schedule information.

6. The computer system of claim 1, wherein the at least one processor is further configured to identify the one or more caregivers to address the determined need of the user.

7. The computer system of claim 1, wherein the at least one processor is further configured to:
generate a smart contract for the generated arrangements with the determined third-party service provider; and
store the smart contract in a blockchain ledger associated with the user, wherein processing for a hash for the blockchain ledger is distributed over multiple computer devices.

8. A computer-implemented method for matching consumers to providers, the method implemented using computer system including at least one processor in communication with at least one memory device and one or more sensors associated with a vehicle of a user, the method comprising:
generating a plurality of driving patterns of behavior of the user from historical vehicle telematics data collected by the one or more sensors including a good driving pattern for the user;
in response to receiving current vehicle telematics data associated with the user, detecting one or more pattern disruptions of the plurality of driving patterns of behavior of the user;
determining a need of the user based upon the one or more pattern disruptions, the determined need addressable by one or more caregivers associated with the user;
in response to determining that none of the one or more caregivers is available to address the determined need of the user, determining a third-party service provider that is able to meet the determined need for the user;
automatically generating arrangements with the determined third-party service provider; and
transmitting the generated arrangements to a computer device associated with the determined third-party service provider.

9. The computer-implemented method of claim 8 further comprising:
receiving registration data from the user; and
generating the plurality of driving patterns of behavior of the user by analyzing the registration data.

10. The computer-implemented method of claim 8 further comprising continuously receiving user data from the one or more sensors, wherein the user data includes the historical vehicle telematics data, and wherein the historical vehicle telematics data includes movement data.

11. The computer-implemented method of claim 8, wherein the one or more pattern disruptions include a negative driving pattern for the user.

12. The computer-implemented method of claim 8 further comprising:
receiving schedule information from the one or more caregivers; and
determining availability of the one or more caregivers based on the received schedule information.

13. The computer-implemented method of claim 8 further comprising identifying the one or more caregivers to address the determined need of the user.

14. The computer-implemented method of claim 8 further comprising:
generating a smart contract for the generated arrangements with the determined third-party service provider; and
storing the smart contract in a blockchain ledger associated with the user, wherein processing for a hash for the blockchain ledger is distributed over multiple computer devices.

15. At least one non-transitory computer-readable medium having computer-executable instructions thereon, wherein when executed by at least one processor of a computer system in communication with one or more sensors associated with a vehicle of a user, the computer-executable instructions cause the at least one processor to:
generate a plurality of driving patterns of behavior of the user from historical vehicle telematics data collected by the one or more sensors including a good driving pattern for the user;
in response to receiving current vehicle telematics data associated with the user, detect one or more pattern disruptions of the plurality of driving patterns of behavior of the user;
determine a need of the user based upon the one or more pattern disruptions, the determined need addressable by one or more caregivers associated with the user;
in response to determining that none of the one or more caregivers is available to address the determined need of the user, determine a third-party service provider that is able to meet the determined need for the user;
automatically generate arrangements with the determined third-party service provider; and
transmit the generated arrangements to a computer device associated with the determined third-party service provider.

16. The least one non-transitory computer-readable medium of claim 15, wherein the computer-executable instructions further cause the at least one processor to:
receive registration data from the user; and
generate the plurality of driving patterns of behavior of the user by analyzing the registration data.

17. The least one non-transitory computer-readable medium of claim 15, wherein the computer-executable instructions further cause the at least one processor to continuously receive user data from the one or more sensors, wherein the user data includes the historical vehicle telematics data, and wherein the historical vehicle telematics data includes movement data.

18. The least one non-transitory computer-readable medium of claim 15, wherein the computer-executable instructions further cause the at least one processor to:
receive schedule information from the one or more caregivers; and
determine availability of the one or more caregivers based on the received schedule information.

19. The least one non-transitory computer-readable medium of claim 15, wherein the computer-executable instructions further cause the at least one processor to identify the one or more caregivers to address the determined need of the user.

20. The least one non-transitory computer-readable medium of claim 15, wherein the computer-executable instructions further cause the at least one processor to:
generate a smart contract for the generated arrangements with the determined third-party service provider; and
store the smart contract in a blockchain ledger associated with the user, wherein processing for a hash for the blockchain ledger is distributed over multiple computer devices.
